(19) United States
(12) Patent Application Publication
(10) Pub. No.: US 2009/0093635 A1
(43) Pub. Date: Apr. 9, 2009

(54) PROCESS FOR MAKING POLYMORPH FROM I OF (S) - (+) -METHYL-ALPHA- (2-CHLOROPHENYL) -6, 7-DYHIDRO-THIENO- [3, 2-c] PYRIDINE-5 (4H) -ACETATE HYDROGEN SULFATE

(76) Inventors: Sandor Garadnay, Esztergom (HU); Istvan Greiner, Budapest (HU); Jozsef Neu, Budapest (HU)

Correspondence Address: K.F. ROSS P.C., 5683 RIVERDALE AVENUE, SUITE 203 BOX 900, BRONX, NY, 10471-0900, US

(21) Appl. No.: 12/282,037

(22) PCT Filed: Mar. 8, 2006
(86) PCT No.: PCT/HU07/00021

§ 371 Date: Nov. 26, 2008

(30) Foreign Application Priority Data
Mar. 9, 2006 (HU) P0600194

(51) Int. Cl. C07D 495/04

(52) U.S. Cl. 546/114

(57) ABSTRACT

The invention relates to a process for the preparation of the pharmaceutically applicable polymorph Form I of (S)-(+)-methyl-α-(2-chlorophenyl)-6,7-dyhidro-thieno[3,2-c]-pyridine-5(4H)-acetate hydrogen sulfate of formula I; by reacting (S)-(+)-methyl-α-(2-chlorophenyl)-6,7-dyhidro-thieno[3,2-c]pyridine-5(4H)-acetate and sulfuric acid in the presence of solvents which comprises dissolving (S)-(+)-methyl-α-(2-chlorophenyl)-6,7-dyhidro-thieno[3,2-c]pyridine-5(4H)-acetate in an ether; mixing this solution with a solution of a C₆-C₁₁ alcohol and sulfuric acid; and recovering the so obtained compound of formula I from the mother liquor.

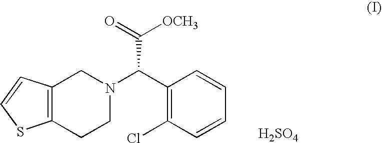

The invention relates to a process for the preparation of the pharmaceutically applicable polymorph Form I of (S)-(+)-methyl-α-(2-chlorophenyl)-6,7-dihydro-thieno[3,2-c]piridine-5(4H)-acetate hydrogen sulfate (also known as (S)-(+)-clopidogrel hydrogen sulfate) of formula (I)

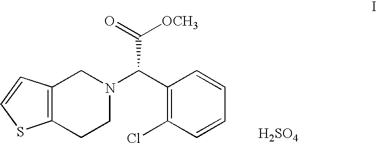

by mixing an ether solution of (S)-(+)-methyl-α-(2-chlorophenyl)-6,7-dihydro-thieno[3,2-c]pyridine-5(4H)-acetate (also known as (S)-(+)-clopidogrel base) with a sulfuric acid solution of a C₆-C₁₁ alcohol, then recovering the precipitated polymorph Form I of (S)-(+)-clopidogrel hydrogen sulfate from the mother liquor.

The (S)-(+)-clopidogrel hydrogen sulfate of formula (I) is a known valuable pharmaceutical substance used as a platelet aggregation inhibitor and anti-thrombotic agent.

The first process for the preparation (S)-(+)-clopidogrel hydrogen sulfate of formula (I) was disclosed in EP 281 459. A large number of chemical processes have been developed for the preparation of the (S)-(+)-clopidogrel hydrogen sulfate of formula (I).

WO 99/65915 published international patent application was the first to state that at least two polymorph forms of the compound of formula (I) exist. The first method for the preparation of polymorph Form II (Melting point: 176±3° C.) was also described. Moreover, it was disclosed that the crystalline form (Melting point: 184±3° C.) described in EP 281,459 corresponds to the polymorph Form I. The above publication discloses the differences between the stabilities, physical properties, spectral characteristics and preparation methods of crystalline Forms I and II.

According to WO 99/65915 published international patent application polymorph Form II is prepared by dissolving (S)-(+)-clopidogrel base in acetone then sulfuric acid (80%) was added at 20° C. According to Example 1B polymorph Form I is prepared almost under the same circumstances. However, according to Example 2 pure polymorph Form II is obtained by seeding the polymorph Form I with polymorph Form II.

One skilled in the art would appreciate that the above WO 99/65915 process for the preparation of polymorph Form I of (S)-(+)-clopidogrel base using acetone as a solvent can not be regarded as susceptible of industrial application since the polymorph Form I undergoes a transformation to polymorph Form II as a consequence of easy formation or potential presence of polymorph Form II.

Numerous further patent applications deal with the preparation of polymorph Form I of compound of formula (I). The polymorph Forms III, IV, V, VI and the amorphous form of (S)-(+)-clopidogrel hydrogen sulfate are disclosed in WO 2003/051362 published patent application wherein clopidogrel hydrogen sulfate Form III is defined as a solvate with 1-butanol, Form IV is a solvate with 2-propanol, Form V is a solvate with 2-butanol and the Form VI is a solvate with 1-propanol.

Besides the polymorph Forms III, IV, V and VI WO 2003/051362 published patent application also discloses a new method for the preparation of the polymorph Form I by adding an antisolvent to a solution of (S)-(+)-clopidogrel hydrogen sulfate in alcohol. Specifically, to a solution of compound of formula (I) in an 1, 3-2fold amount of methanol a 10-150fold excess of methyl-t-butyl ether or diethyl ether was added whereby polymorph Form I is precipitated. The polymorph Form I can be obtained also by adding 9fold amount of methyl-t-butyl ether or diethyl ether to a solution of compound of formula (I) in 3fold amount of ethanol.

In the course of our experiences the methods for the preparation of the polymorph Form I described in WO 2003/051362 were accomplished. As a result we have found that in the case of an insufficient dissolving of (S)-(+)-clopidogrel hydrogen sulfate, the undissolved part of (S)-(+)-clopidogrel hydrogen sulfate acts as seed during the precipitating process. Hereby there is a possibility to form predominantly polymorph Form II. According to the above method the forming of polymorph form is uncertain. Because of this disadvantage of the procedure the method can not to be taken in account as an industrial procedure.

WO 2003/051362 published patent application also discloses a new method for the preparation of polymorph Form II of compound of formula (I) by adding sulfuric acid (80%) to a solution of (S)-(+)-clopidogrel base in methyl-ethyl-ketone, dichloromethane, toluene, chloroform, ethyl-acetate, methyl-t-butyl ether or 1,4-dioxane at a temperature of 20° C. In another reaction route the polymorph Form II was obtained from a solution of (S)-(+)-clopidogrel hydrogen sulfate in acetonitrile by adding diethyl ether to it.

WO 2004/020443 published patent application is directed to a method for preparing polymorph Form I of compound of formula (I) by dissolving (S)-(+)-clopidogrel base in a C₁-C₅ alcohol, preferably in 2-propanol or in an ester thereof, preferably butyl-acetate. Then the solution was cooled to a temperature between (−5°)-0° C. and sulfuric acid (98%) was added. For seeding crystals of polymorph Form I of (S)-(+)-clopidogrel hydrogen sulfate was added. In a further procedure, (S)-(+)-clopidogrel base was dissolved in a mixture of 2-propanol and butyl-acetate then sulfuric acid (98%) was added at a temperature between (−2)-0° C. without seeding. In each of the above procedures polymorph Form II was also recovered from the mother liquor.

One of the significant disadvantages of the procedure is the presence of polymorph Form II in the mother liquor which makes doubtful the industrial applicability of the procedure. Besides, the chance of forming of polymorph Form IV disclosed originally in WO 2003/051362 is increased by using 2-propanol. In addition by using a seed in inappropriate polymorph purity the chance of formation of polymorph Form II is expands.

In a process disclosed in WO 2004/020443 published patent application polymorph Form I of compound of formula (I) was obtained from (S)-(+)-clopidogrel hydrogen sulfate by heating it in a 46fold amount of butyl-acetate to reflux temperature then, after cooling the polymorph Form I was obtained. However, by using such a high solvent:product ratio the equipments are underutilized, and therefore the procedure is insufficient for industrial application.

According to WO 2004/048385 published patent application polymorph Form I of compound of formula (I) was prepared by adding concentrated sulfuric acid to a solution of (S)-(+)-clopidogrel base in dioxan, 1,2-dimethoxyethane, bis(2-ethoxyethyl ether), or in a mixture of methyl t-butyl ether/2-propanol or in isobutyl methyl ketone.

However, the method described in WO 2004/048385 is contradictory with the data of the former WO 2003/051362 published international patent application, wherein polymorph Form II was obtained by using similar solvents. Our experiments also confirm the data published in WO 2003/051362.

According to another procedure disclosed in WO 2004/048385 published patent application the (S)-(+)-clopidogrel base was dissolved in ethanol or methoxyethanol then sulfuric acid (96%) was added and the product was precipitated by adding 9fold amount of methyl t-butyl ether or dimethoxyethane.

Disadvantage of the above method, similarly to the procedure described in WO 2003/051362, is that the precipitation may result in oily (S)-(+)-clopidogrel hydrogen sulfate, which is against of the industrial application of the procedure.

WO 2004/081016 published patent application discloses a process for the preparation of polymorph Form I by adding sulfuric acid (80%) to a solution of (S)-(+)-clopidogrel base in acetone at a temperature of 20° C. and subsequent stirring at a temperature of −20° C., or by adding sulfuric acid (80%) to a solution of (S)-(+)-clopidogrel base in acetone at a temperature of between 50-52° C. To perform the above process under industrial conditions is very dangerous while the yields are below 62%. With full knowledge of the processes described in WO 99/65915 published international patent application wherein the processes were performed under similar circumstances with inconsistent results, the above method seems to be not suitable for the preparation of polymorph Form I in an industrial scale.

According to WO 2004/081016 published international patent application the polymorph Form II was prepared from amorphous form of (S)-(+)-clopidogrel hydrogen sulfate by stirring it in acetone or in ethyl acetate.

According to a process disclosed in WO 2005/003139 published international patent application the polymorph Form I of the compound of formula (I) was obtained by adding sulfuric acid (96%) to a solution of (S)-(+)-clopidogrel base in acetone, dichloromethane or in 2-propanol at a temperature of 10-15° C. Then the polymorph Form I was precipitated with 25-30fold amount of diisopropyl ether, cyclohexane or ethyl acetate containing 30 weight % of (S)-(+)-clopidogrel hydrogen sulfate polymorph Form I based on the weight of clopidogrel base at a temperature of 0° C.

According to another reaction route a mixture of sulfuric acid (96%) and diisopropyl ether was added at a temperature of 0° C. to a solution of (S)-(+)-clopidogrel hydrogen sulfate polymorphic Form I in a 25fold amount of diisopropyl ether and 4fold amount of 2-propanol containing the polymorph Form I in an amount of 30 weight % based on the weight of clopidogrel base.

A serious disadvantage of the procedure described in WO 2005/003139 published international patent application is that polymorph Form I seed crystals are used in very large quantities. Besides, according to the procedure described in WO 2003/051362 published international patent application the polymorph Form II was obtained by using similar solvents; consequently, the method described in WO 2005/003139 is unsuitable for industrial purposes.

WO 2005/003139 published international patent application discloses a method for the preparation of the polymorph Form II of compound of formula (I), in which a solution of (S)-(+)-clopidogrel base in acetone, dichloromethane, or in a mixture of ethyl acetate/acetone, dichloromethane/acetone or methyl ethyl ketone/acetone was seeded with polymorph Form I then concentrated sulfuric acid was added to precipitate polymorph Form II.

Both our reproduction studies and the data presented in WO 2005/003139 published patent application showed that by using solvents generally applied for the preparation of polymorph Form I, even in the presence of polymorph Form I seed crystals the polymorph Form II was often obtained.

According to the disclosure of WO 2005/003139 published patent application by adding sulfuric acid to a solution of (S)-(+)-clopidogrel hydrogen sulfate in dichloromethane then precipitating the product without seed crystals by diisopropyl ether, the amorphous form of (S)-(+)-clopidogrel hydrogen sulfate was obtained, showing, that while the use of seed crystals is essential, polymorph form of the product is not easy-to-foresee.

According to a process described in WO 2005/012300 published international patent application the polymorph Form I of the compound of formula (I) was obtained by dissolving (S)-(+)-clopidogrel base in ethyl acetate, and to the resultant solution seed crystals of polymorph Form I (2.5% based on the weight of clopidogrel base) and concentrated sulfuric acid were added.

The yield is however is below 70%. Besides, the procedure described in the above WO 2005/012300 published international patent application is inconsistent with the disclosure of WO 2003/051362 published international patent application since according to the latter patent application polymorph Form II was obtained by using ethyl acetate.

According to WO 2005/016931 published international patent application polymorph Form I of the compound of formula (I) was obtained by dissolving (S)-(+)-clopidogrel base in 2-propanol. Then, first a mixture of sulfuric acid (97%) and 2-propanol, then seed crystals of polymorph Form I were added to the solution.

The disadvantage of the above procedure is that the use of 2-propanol results in an increased risk of formation of polymorph Form IV as described in WO 2003/051362 published international patent application.

In the other reaction route described in WO 2005/016931 published international patent application (S)-(+)-clopidogrel hydrogen sulfate was dissolved in a mixture of methanol and 2-propanol then crystallization was performed.

According to Example 4 in WO 2005/016931 published international patent application (S)-(+)-clopidogrel hydrogen sulfate was dissolved in methanol and the resultant solution was evaporated then the residue was crystallized from 2-propanol.

In Example 6 of WO 2005/016931 published international patent application polymorph Form II was obtained by dissolving (S)-(+)-clopidogrel hydrogen sulfate in a mixture of methanol/2-propanol then a crystallization was performed.

In Examples 4 and 6 in WO 2005/016931 published international patent application either polymorph Form I or polymorph Form II can be obtained depending on that the reaction temperature applied was between 10-12° C. or was kept at 25° C. A further disadvantage is that the method goes with poor yields (less than 70%). The improper dissolution of (S)-(+)-clopidogrel hydrogen sulfate may cause another problem since the undissolved microcrystals remained in the solution may act as seed crystals decreasing the chance of forming polymorph Form I. Therefore the above procedure is not suitable for industrial purposes.

In a process disclosed in 2005/059696 US patent application (S)-(+)-clopidogrel base was dissolved in 2-butanol, then sulfuric acid was added, but the yield is only 70%. A further disadvantage of the procedure is the severe risk of formation of polymorph Form V (a solvate with 2-butanol) as described in WO 2003/051362 published international patent application.

According to WO 2005/063708 published international patent application the polymorph Form I of compound of formula (I) was prepared by dissolving (S)-(+)-clopidogrel base in n-hexanol or in n-heptanol, to the solution aqueous sulfuric acid was added, than seeded with polymorph Form I.

Our investigations applying the above procedure showed that when polymorph Form II was present in many instances the polymorph Form II was obtained. Besides, improper addition of the sulfuric acid may result in oily precipitated material, thus the above procedure is not suitable for industrial purposes.

According to another method for the preparation of polymorph Form I described in WO 2005/063708 (S)-(+)-clopidogrel base was dissolved in a mixture of n-hexanol/water or n-decanol/water, then concentrated sulfuric acid was added and it was seeded by polymorph Form I.

In this procedure the seed crystals raise the risk of forming of polymorph Form II. Besides, another disadvantage of the process is the slow hydrolyzation of methyl ester group of (S)-(+)-clopidogrel to carbonic acid under the influence of the water applied in acidic medium in the procedure, whereby the purity of product is worsen. Therefore the so obtained product is unsuitable for therapeutic use.

According to a further procedure described in WO 2005/063708 published international patent application the polymorph Form I was prepared by dissolving (S)-(+)-clopidogrel hydrogen sulfate in hexanol, then the resultant precipitate was stirred for 12 hours, filtered and washed with methyl t-butyl ether. In another reaction route (S)-(+)-clopidogrel base was dissolved in n-decanol, then aqueous sulfuric acid was added to the reaction mixture. The resulting precipitate was stirred for 24 hours and washed with methyl t-butyl ether.

A drawback of this procedure is that the yield is very low, about 60%. The procedure is also disadvantageous from the environmental point of view due to the long carbon chain alcohols present in a 7fold amount. It is hard to decrease the amount of alcohols with high boiling points in the end product under the solvent limit required by the pharmaceutical authority.

According to WO 2005/100364 published international patent application the preparation of polymorph Form I of compound of formula (I) was carried out by dissolving (S)-(+)-clopidogrel base in methyl acetate or ethyl acetate and adding sulfuric acid (90-95% or concentrated) at a temperature between −15° C. and −5° C.

A drawback of the above process is that the use of low chain acetic acid esters may result in the formation of polymorph Form II as it was described in the former WO 2003/051362 published international patent application, which observation is inconsistent with the disclosure of WO 2003/051364 published international patent application. Our experiments also confirm the disclosure of WO 2005/100362.

In another reaction route disclosed in WO 2005/100364 published international patent application to a methyl t-butyl ether solution of (S)-(+)-clopidogrel base methyl t-butyl ether containing sulfuric acid (40%) was added at a temperature between −15° C. and −5° C. to obtain polymorph Form I.

WO 2003/051362 and WO 2005/063708 published international patent applications also contain methods for the preparation of polymorph Form II in pure methyl t-butyl ether; this is confirmed by our experiments, too. Accordingly, the above procedure seems to be improper for the preparation of polymorph Form I.

WO 2005/100364 published international patent application discloses further methods for the preparation of polymorph Form I of compound of formula (I). Accordingly, concentrated sulfuric acid was added at a temperature between −5° C. and 5° C. to a solution of (S)-(+)-clopidogrel base in diisopropyl ether. According to the other procedure described in the patent application dichloromethane containing 10% sulfuric acid was added at a temperature between −15° C. and −5° C. to a solution of (S)-(+)-clopidogrel base in a mixture of methyl acetate/dichloromethane or ethyl acetate/diisopropyl ether. In the course of our experiments we have found that performing the procedures for the preparation of polymorph Form I according to the methods described in the aforesaid patent applications often the polymorph Form II was obtained as a consequence of the use of improper solvent.

As it was mentioned for the preparation of polymorph Form I of (S)-(+)-clopidogrel hydrogen sulfate of formula (I) large number of chemical processes have been developed. However data presented in the cited references contradict to each other. Due to their irreproducibility which occurs particularly in the presence of polymorph Form II, the methods described in the above published patent applications are unsuitable for the industrial production of polymorph Form I of (S)-(+)-clopidogrel hydrogen sulfate.

All the preparation processes of polymorph Form I of compound of formula (I) known in the technical literature have the following common disadvantages:

the methods are unsuitable for industrial purposes since it is uncertain whether or not the pure polymorph Form I is obtained;

the crystallization step is carried out in large excess of dangerous and environmentally undesirable solvents;

in some cases the seed crystals are used in very large amounts which on one hand is uneconomical and on the other hand the polymorphic purity is worsen;

microcrystals of compound of the formula (I) remained undissolved in the solution may act as seed crystals;

some of the processes can be carried out only at law temperatures which makes the industrial applicability difficult;

data published in some of the cited references are inconsistent to each other hereby illustrating that the problem of preparation of polymorph Form I of compound of formula (I) have not been solved;

due to the low yield the industrial production is uneconomical;

in some processes oily product may separate from the reaction mixture hereby the industrial production becomes impossible;

in some processes aqueous reaction mixture is applied and due to the presence of water in acidic medium the methyl ester group of (S)-(+)-clopidogrel my undergo hydrolysis giving carbonic acid. As a result the purity of the resultant product is reduced and consequently it is not suitable for pharmaceutical use.

Accordingly, in the light of the technical literature the preparation of polymorph Form I of compound of formula (I) seems to be impossible with the desired certainty at industrial scale.

Our aim was to provide an economical process by which the polymorph Form I of (S)-(+)-clopidogrel hydrogen sulfate can be prepared in the required drug-purity via reaction steps producing good yields and besides the formation of polymorph Form I is suitably assured.

The invention is based on the recognition, that when (S)-(+)-clopidogrel base is dissolved in an ether and mixed with a solution of C₆-C₁₁ alcohol in sulfuric acid, polymorph Form I of (S)-(+)-clopidogrel hydrogen sulfate is obtained.

The differences between the process according to the invention and the processes described in the technical literature are as follows.

Comparing the process described in WO 2003/051362 published international patent application with the process according to the invention the difference is that for the preparation of polymorph Form I of compound of formula (I) in the process according to the invention (S)-(+)-clopidogrel base and longer chain (C₆-C₁₁) alcohols are applied instead of (S)-(+)-clopidogrel hydrogen sulfate and lower alcohols (methanol or ethanol).

A further difference is that in the procedure according to the invention by adding sulfuric acid after the dissolution of (S)-(+)-clopidogrel base in ether in the presence of a C₆-C₁₁ alcohol the polymorph Form I of compound of formula (I) is obtained instead of polymorph Form II of compound of formula (I).

Contrary to the process according to the invention in the method described in WO 2004/020443 published international patent application (S)-(+)-clopidogrel base is dissolved in an alcohol instead of ether, and the alcohol is a C₁-C₅ alcohol instead of a C₆-C₁₁ alcohol and no ether is applied.

Comparing the processes disclosed in the present patent application with WO 2005/016931 it can be seen, that in the procedure according to the invention (S)-(+)-clopidogrel base is dissolved in an ether then, a mixture of a C₆-C₁₁ alcohol and sulfuric acid was added to the solution. In contract, WO 2005/016931 published international patent application claims the process wherein a solution of (S)-(+)-clopidogrel base in a C₃-C₁₀ alcohol is reacted with sulfuric acid, but ether is not applied to dissolve the (S)-(+)-clopidogrel base. Further, the use of long chain alcohols to dissolve the clopidogrel base is not exemplified, example only for 2-propanol is given.

Contrary to the process according to the invention, in the procedure disclosed in WO 2005/063708 published international patent application (S)-(+)-clopidogrel base, C₆-C₁₂ alcohol and diluted sulfuric acid are reacted to give polymorph Form I of compound of formula (I); then the mixture is filtered, washed with methyl t-butyl ether. Accordingly, the reaction mixture described in the above-cited reference contains water, while ether is not applied in said procedure.

Contrary to the process according to the invention in the process disclosed in WO 2005/100364 published international patent application a solution of (S)-(+)-clopidogrel base in methyl t-butyl ether is reacted with sulfuric acid without using an alcohol.

In the course of our experiments we have surprisingly found, that when the (S)-(+)-clopidogrel base is dissolved in a solvent known as an antisolvent, i.e. in an ether and to this solution a mixture of a long chain alcohol and sulfuric acid is added, the resulting product was identified as polymorph Form I of compound of formula (I) by DSC, TGA, IR, and XRPD.

This is more surprising as in the Examples of the former processes for the preparation of polymorph forms of compound of formula (I) the use of solvents similar to those used in the present invention are described, but not the polymorphic Form I is obtained. For example as it is written in Example 10 of WO 2003/051362 literature, (S)-(+)-clopidogrel base was dissolved in methyl t-butyl ether, then sulfuric acid was added and the resulting product was identified as polymorph Form II. Further, according to Example 23 of WO 2005/063708 reference by mixing the amorphous form in methyl t-butyl ether also polymorph Form II is obtained.

In the course of our initial experiments performing the reproduction of the above known preparation processes it has been established, that when the dissolution of (S)-(+)-clopidogrel hydrogen sulfate is incomplete, the polymorph Form II can be obtained since the undissolved part of (S)-(+)-clopidogrel hydrogen sulfate acts as seed crystal during the precipitation procedure.

In the course of our further experiments a new process has been elaborated to give the polymorph Form I of compound of formula (I) in a very safe and at industrial scale easy-to-perform manner.

On the basis of our above experiments we have surprisingly found, that the process according to the invention provides such a high safety for the preparation of polymorph Form I of (S)-(+)-clopidogrel hydrogen sulfate that polymorph Form I is obtained even if the amount of polymorph Form II being always present in traces was increased intentionally.

Since the process according to the invention is performed without water, no carbonic acid impurity can be formed by hydrolization of the ester group; i.e. the level of impurities in the resultant product is not increased.

In our process according to the invention the polymorph Form I of (S)-(+)-clopidogrel hydrogen sulfate can be obtained in very high purity and yield; and both the yield and purity are better than those of the products described in the technical field.

The preparation process according to the invention becomes more economical by using an easy-to-recirculate ether and a small amount of a high chain alcohol.

Taking into consideration the above technical literature we can safety say that the polymorph Form I of compound of formula I has not been prepared by the process according to the invention.

The invention relates to a process for the preparation of the pharmaceutically applicable polymorph Form I of (S)-(+)-methyl-α-(2-chlorophenyl)-6,7-dihydrothieno[3,2-c]pyridine-5(4H)-acetate hydrogen sulfate of formula (I) by reacting (S)-(+)-methyl-α-(2-chlorophenyl)-6,7-dihydrothieno[3,2-c]-pyridine-5(4H)-acetate with sulfuric acid in the presence of solvents, characterized in that (S)-(+)-methyl-α-(2-chlorophenyl)-6,7-dihydrothieno[3,2-c]piridine-5(4H)-acetate is dissolved in an ether; this solution is mixed with a sulfuric acid solution of a C₆-C₁₁ alcohol; and the crystalline product so obtained is recovered from the mother liquor.

### DETAILED DESCRIPTION OF THE INVENTION

Definitions of terms and abbreviations used herein have the following meanings.

As used herein the term “at room temperature” refers to temperatures between about 20° C. and 25° C.

As used herein the abbreviation “m.p.” refers to melting point.

As used herein the abbreviation “DSC” means differential scanning calorimetric test.

As used herein the abbreviation “TGA” means thermogravimetric analysis.

As used herein the abbreviation “IR” means infrared spectra.

As used herein the abbreviation “PXRD” means powder X-ray diffraction test.

Typically, the preparation process of the therapeutically applicable polymorph Form I of compound of the formula (I) according to the invention can be performed in the following manner. Accordingly, to a sulfuric acid solution of a C₆-C₁₁ alcohol a solution of (S)-(+)-clopidogrel base in an ether is added. After stirring the mixture for 24-48 hours at room temperature the resultant polymorph Form I of (S)-(+)-clopidogrel hydrogen sulfate is separated from the mother liquor. The order of the additions of the two above solutions is interchangeable.

In the process according to the invention as a solvent for the dissolution of (S)-(+)-clopidogrel base one or more, straight or branched chain aliphatic ether of general formula Cₙ—O—Cₘ is used, wherein n and m may be different or identical and n is 1-4, and m is 2-4; preferably methyl t-butyl ether is applied.

In the process according to the invention as C₆-C₁₁ alcohol one or more straight or branched chain, aliphatic or cyclic, primary, secondary or tertiary alcohol, preferably 1-decanol is employed.

In the process according to the invention the (S)-(+)-clopidogrel base is dissolved in 0.7-7.4fold, preferably 7.4fold amount of ether.

In the process according to the invention the alcohol is applied in a 0.8-4.1fold, preferably 1.66fold amount based on the weight of the (S)-(+)-clopidogrel base.

In the process according to the invention the ether is applied in a 0.17-8.96fold, preferably 4.45fold amount based on the amount of the alcohol used in the procedure.

In the process according to the invention 0.9-1.25 mol equivalents, preferably 1 mol equivalent sulfuric acid is used per one mol of (S)-(+)-clopidogrel base.

The concentration of the sulfuric acid used in the preparation method according to the invention is 90-100 wt %, preferably 96 wt %.

In the course of the procedure according to the invention the separate preparation of the reagents is very important; namely, the solution of the (S)-(+)-clopidogrel base in an ether and the sulfuric acid solution of a C₆-C₁₁ alcohol must be separately prepared. Besides the ratio of the reagents is also very important in the process according to the invention.

The advantage of the invention appears in that the ratio of the solvents to each other may vary between broad limits.

The use of the alcohol in a large amount (4.1fold amount based on the weight of (S)-(+)-clopidogrel base) and the ether in a small amount (0.73fold amount based on the weight of the (S)-(+)-clopidogrel base) results in low yield as it is shown in Example 1.

When the order of addition of the two solutions used in the procedure is reversed, i.e. the acidic alcohol is added to the solution of (S)-(+)-clopidogrel base in ether, a lower yield is obtained too, as it shown in Example 15.

In the process according to the invention the crystallization is carried out preferably for 24-48 hours.

For the preparation of polymorphic Form I of compound of formula (I) the reaction is performed preferably at room temperature.

In the process according to the invention the yield is over 80%, moreover at best is almost 90%.

The process according to the invention has the advantage that based on the weight of the (S)-(+)-clopidogrel base only a 1.66fold amount of the high chain alcohol is necessary to obtain a yield of about 90%.

The solvent mixture used in the process according to the invention is more advantageous also from the environmental point of view since the ether component of the solvent mixture can be recovered by a simple distillation owing to the great difference between the boiling points and the ether recovered may be re-used again.

In the preparation process according to the invention water is not employed therefore the product does not contain carbonic acid, a possible impurity when ester hydrolysis can takes place.

Since the high chain alcohol is used in small amount and the ether can be recirculated, the process according to the invention is economical.

The shorter is the carbon chain of the alcohol used, the higher is the solubility of (S)-(+)-clopidogrel hydrogen sulfate, and increased solubility goes with loss of yield as shown in Example 9.

Applying the process according to the invention for the preparation of polymorph Form I S)-(+)-clopidogrel hydrogen sulfate of formula (I), there is no need to employ seed crystals.

A major advantage of the process according to the invention is that the polymorph Form I of (S)-(+)-clopidogrel hydrogen sulfate is formed with great certainty even in the presence of polymorph Form II of (S)-(+)-clopidogrel hydrogen sulfate impurity. It is shown by Example 13, wherein polymorph Form I was obtained in spite of intentional addition of polymorph Form II of (S)-(+)-clopidogrel hydrogen sulfate to the reaction mixture.

In the process according to the invention neither the manner of stirring nor the reaction temperature are not critical.

Advantages of the process according to the invention are as follows:

high degree of polymorph purity;

safe industrial applicability;

high yield;

decrease of undesirable environmental effects;

less contamination in the product;

employing of seed crystals is not necessary;

the manner of stirring is not critical;

the temperature employed is not critical;

sulfuric acid of 90-100 wt % can be used;

the ratio of the solvents used may vary between broad limits.

The invention is illustrated by the following non-limiting Examples.

### EXAMPLE 1

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.54 ml of sulfuric acid (96 wt %) was added to 15 ml (12.4 g) of 1-decanol under stirring, then a solution of 3.0 g (S)-(+)-clopidogrel base in 3.0 ml (2.2 g) methyl t-butyl ether was added under room temperature for about 15 minutes while a hard-to-stir material separates out. Under room temperature the reaction mixture was stirred for another 48 hours then it was filtered and washed with methyl t-butyl ether and dried in vacuum at a temperature between 50-60° C. 2.2 g of compound of formula (I) was obtained which was identified as polymorph Form I on the basis of analytical test data.

Yield: 56.0%.

Melting point: 180-181° C. (determined by DSC).

### EXAMPLE 2

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.72 ml (96 m/m %) of sulfuric acid was added to 4 ml (3.3 g) of 1-decanol with stirring. Then a solution of 4.0 g (S)-(+)-clopidogrel base in 20.0 ml (14.8 g) of methyl t-butyl ether was added under room temperature for about 15 minutes meanwhile a hard-to-stir material separates out. Under room temperature the reaction mixture was stirred for another 26 hours then it was filtered and washed with methyl t-butyl ether. It was dried in vacuum at a temperature between 50-60° C. yielding 4.58 g of compound of formula (I) which was identified as polymorph Form I on the basis of analytical test data.

Yield: 87.7%.

Melting point: 180-181° C. (determined by DSC).

### EXAMPLE 3

### Preparation of polymorph Form I of (S)-(+)-methyl-α-(2-chlorophenyl)-6,7-dyhidro-thieno[3,2-c]pyridine-5(4H)-acetate hydrogen sulfate of formula I

The procedure described in Example 1 was carried out except that instead of the amounts used in Example 1, 4 ml (3.3 g) of 1-decanol, 0.68 ml (96 wt %) of sulfuric acid, 4.0 g of (S)-(+)-clopidogrel base, and 40.0 ml (29.6 g) of methyl t-butyl ether were used. In the above manner 4.52 g of compound of formula (I) was obtained which was identified as polymorph Form I on the basis of analytical test data.

Yield: 86.5%.

Melting point: 182-183° C. (determined by DSC).

### EXAMPLE 4

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.72 ml (96 wt %) sulfuric acid was added to 8 ml (6.6 g) of 1-decanol with stirring. Then a solution of 4.0 g (S)-(+)-clopidogrel base in 40.0 ml (29.0 g) of diisopropyl ether was added under room temperature for about 15 minutes meanwhile a hard-to-stir material separates out. Under room temperature the reaction mixture was stirred for another 26 hours then it was filtered and washed with diisopropyl ether and dried under vacuum at a temperature between 50-60° C. In this manner 4.66 g of compound of formula (I) was obtained which was identified as polymorph Form I on the basis of analytical test data.

Yield: 89.3%.

Melting point: 180-181° C. (determined by DSC).

### EXAMPLE 5

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.54 ml of sulfuric acid (96 wt %) was added to 6 ml (5.0 g) of 1-decanol with stirring. Then a solution of 3.0 g of (S)-(+)-clopidogrel base in 30.0 ml (21.2 g) of diethyl ether was added under room temperature for about 15 minutes meanwhile a hard-to-stir material separates out. Under room temperature the reaction mixture was stirred for another 48 hours then filtered, washed with diethyl ether and dried under vacuum at a temperature between 50-60° C. In this manner 3.29 g compound of formula (I) was obtained which was identified as polymorph Form I on the basis of analytical test data.

Yield: 84.0%.

Melting point: 184-185° C. (determined by DSC).

### EXAMPLE 6

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 1.8 ml of sulfuric acid (96 wt %) was added to 20 ml (16.6 g) of 1-decanol with stirring. Then a solution of 10.0 g (S)-(+)-clopidogrel base in 100 ml (74.0 g) of methyl t-butyl ether was added under room temperature for about 15 minutes meanwhile a hard-to-stir material separates out. Under room temperature the reaction mixture was stirred for another 48 hours then filtered and washed with methyl t-butyl ether and dried under vacuum at a temperature between 50-60° C. In the above matter 11.5 g of compound of formula (I) was obtained which was identified as polymorph Form I on the basis of analytical test data.

Yield: 88.1%.

Melting point: 184-185° C. (determined by DSC).

For the analytical characterization of the thus prepared compounds Shimadzu DSC-50 Calorimeter, TA Instruments DSC Q10 and TG Q50, Nicolet 380 FTIR Spectrophotometer and Philips PW 1840 X-ray diffraction instruments were used.

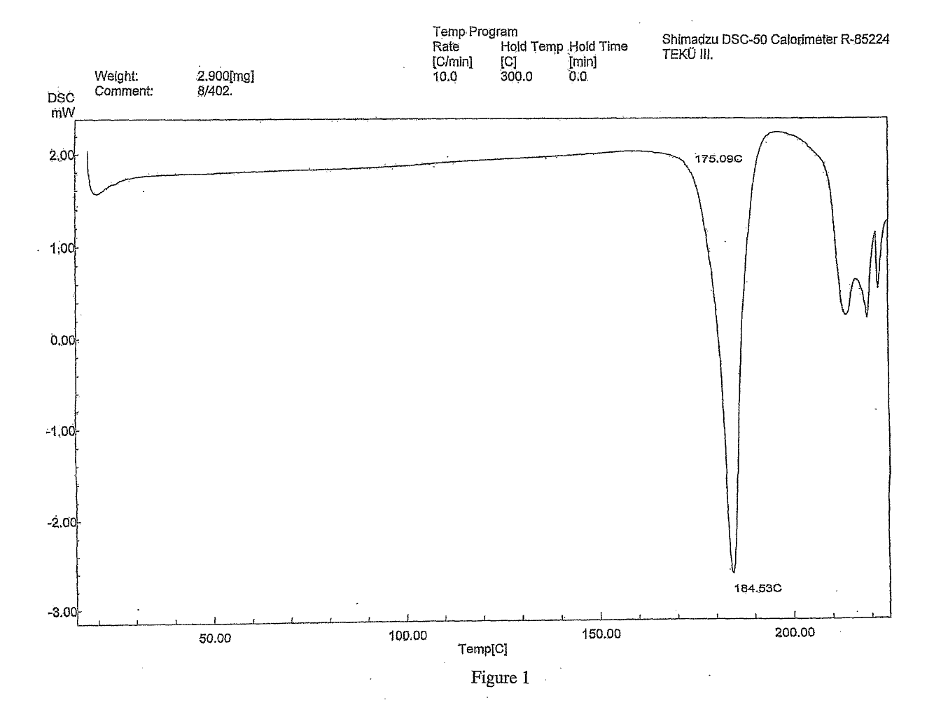

To support the process according to the invention FIGS. 1-4 were enclosed. Brief description of FIGS. 1-4 is as follows:

FIG. 1 represents a differential scanning calorimetric (DSC) thermogram of polymorph Form I of (S)-(+)-methyl-α-(2-chlorophenyl)-6,7-dyhidro-thieno[3,2-c]pyridine-5(4H)-acetate hydrogen sulfate of formula I prepared according to Example 6.

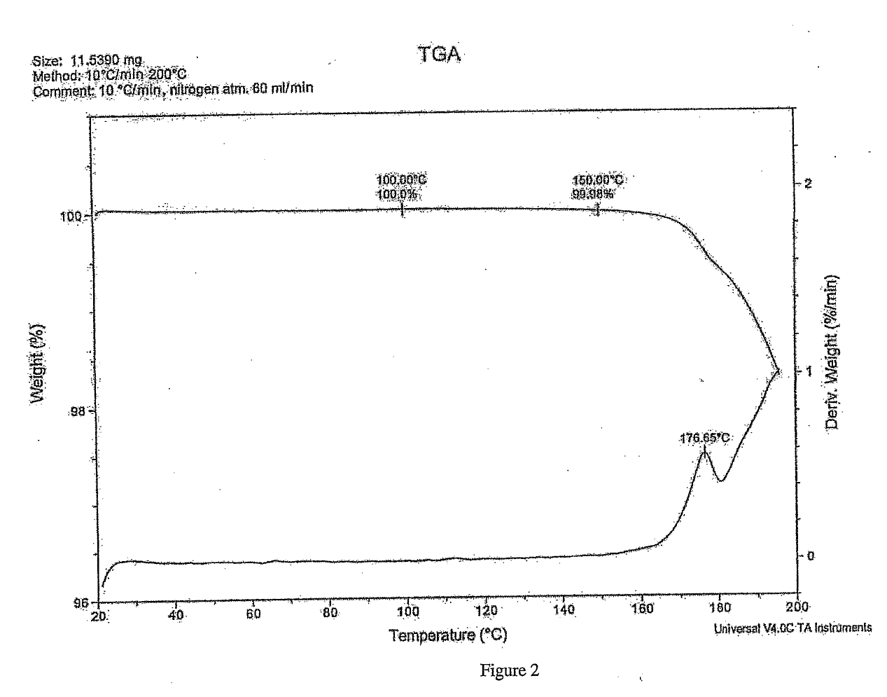

FIG. 2 shows thermogravimetric analysis (TGA) test data of polymorph Form I of (S)-(+)-methyl-α-(2-chlorophenyl)-6,7-dyhidro-thieno[3,2-c]pyridine-5(4H)-acetate hydrogen sulfate of formula I prepared according to Example 6.

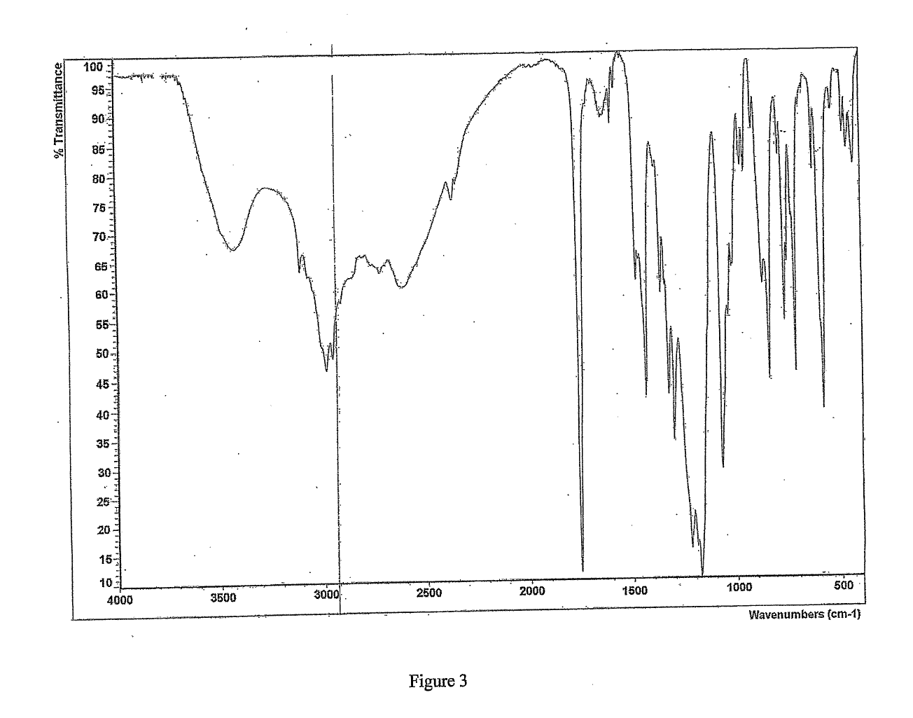

FIG. 3 represents an infrared (IR) spectrum of polymorph Form I of (S)-(+)-methyl-α-(2-chlorophenyl)-6,7-dyhidro-thieno[3,2-c]pyridine-5(4H)-acetate hydrogen sulfate of formula I prepared according to Example 6.

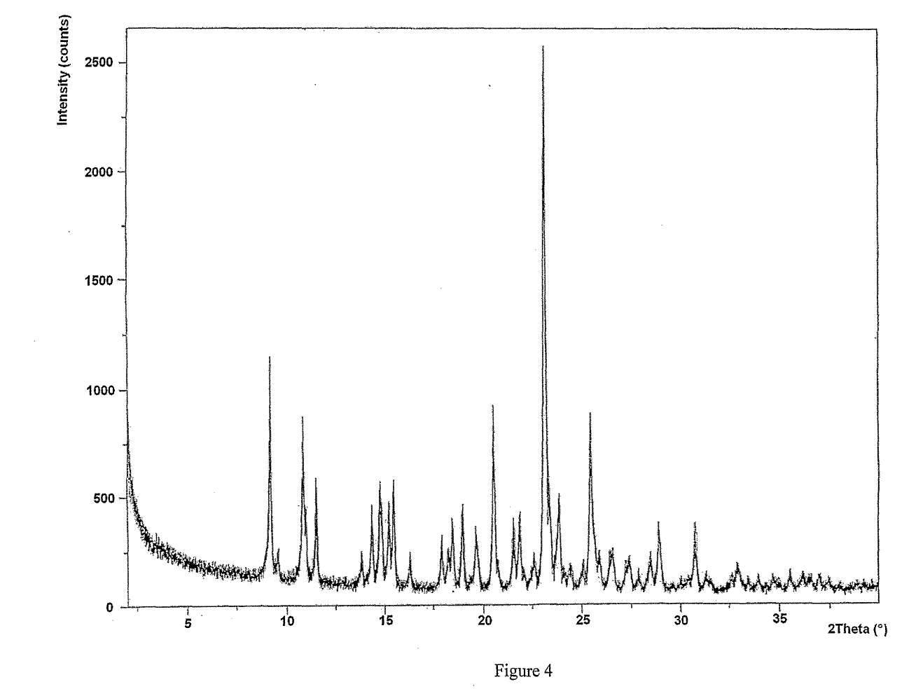

FIG. 4 shows a powder X-ray diffraction (PXRD) pattern of polymorph Form I of (S)-(+)-methyl-α-(2-chlorophenyl)-6,7-dyhidro-thieno[3,2-c]pyridine-5(4H)-acetate hydrogen sulfate of formula I prepared according to Example 6.

### EXAMPLE 7

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.54 ml (96 wt %) of sulfuric acid was added to 6 ml (5.0 g) of 1-nonanol with stirring. Then a solution of 3.0 g (S)-(+)-clopidogrel base in 30.0 ml (22.2 g) of methyl t-butyl ether was added under room temperature for about 15 minutes meanwhile a hard-to-stir material precipitates. Under room temperature the reaction mixture was stirred for another 48 hours and filtered, washed with methyl t-butyl ether and dried under vacuum at a temperature between 50-60° C. In the above manner 3.36 g compound of formula (I) was obtained. Analytical data confirm that the compound is the polymorph Form I.

Yield: 85.8%.

Melting point: 182-183° C. (determined by DSC).

### EXAMPLE 8

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure described in Example 7 the compound of formula (I) was obtained with the difference that instead of 1-nonanol 6 ml (5.0 g) of 1-undecanol was used. In this manner 3.44 g of compound of formula (I) was obtained which was identified by analytical tests as polymorph Form I.

Yield: 87.9%.

Melting point: 182-183° C. (determined by DSC).

### EXAMPLE 9

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure described in Example 7 the compound of formula (I) was obtained except that instead of 1-nonanol 6 ml (4.9 g) of 1-heptanol was used. In this manner 2.63 g of compound of formula (I) was obtained which was identified by analytical tests as polymorph Form I.

Yield: 67.2%.

Melting point: 183-184° C. (determined by DSC).

### EXAMPLE 10

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure according to Example 7 the compound of formula (I) was obtained except that instead of 1-nonanol 6 ml (5.7 g) of cyclohexanol was used. In this manner 3.25 g of compound of formula (I) was obtained which was identified by analytical test as polymorph Form I.

Yield: 83.0%.

Melting point: 181-182° C. (determined by DSC).

### EXAMPLE 11

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure described in Example 1 the compound of formula (I) was obtained except that instead of the amounts used in the procedure of Example 1, 6 ml (5.0 g) of 1-decanol, 0.59 ml (90 m/m %) of sulfuric acid, 3.0 g of (S)-(+)-clopidogel base and 30.0 ml (22.2 g) of methyl-t-butyl ether were used. In this manner 3.55 g of compound of formula (I) was obtained which was identified by analytical tests as polymorph Form I.

Yield: 89.4%.

Melting point: 182-183° C. (determined by DS C).

### EXAMPLE 12

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure described in Example 11 the compound of formula (I) was obtained with the alterations that instead of 0.59 ml (90 wt %) of sulfuric acid 0.54 ml (96 wt %) of sulfuric acid was used furthermore the addition of the solution of clopidogrel base in ether and the stirring of the reaction mixture was at a temperature between 30-35° C. for 48 hours. In this manner 3.29 g of the title compound was obtained. Analytical tests confirmed that the product was polymorph Form I.

Yield: 84.0%.

Melting point: 181-182° C. (determined by DSC).

### EXAMPLE 13

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.51 ml (96 wt %) of sulfuric acid was added to 6 ml (5.0 g) of 1-decanol with stirring. To a solution of 3.0 g of (S)-(+)-clopidogrel base in 30.0 ml (22.2 g) of methyl t-butyl ether 0.05 g of (S)-(+)-clopidogrel hydrogen sulfate polymorph Form II was added then the resulting suspension was added to the above sulfuric acid solution of the 1-decanol under room temperature for about 15 minutes. At this time a hard-to-stir material separated out from the reaction mixture. Under room temperature the reaction mixture was stirred for another 48 hours then it was filtered, washed in methyl t-butyl ether and dried under vacuum at a temperature between 50-60° C. In this manner 3.19 g of product was obtained which was identified as polymorph Form I by analytical tests.

Yield: 81.4%.

Melting point: 181-182° C. (determined by DSC).

### EXAMPLE 14

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Under room temperature 0.67 ml (96 wt %) of sulfuric acid was added to 3.75 ml (3.1 g) of 1-decanol with stirring. To a solution of 3.75 g of (S)-(+)-clopidogrel base in 18.8 ml (13.9 g) of methyl t-butyl ether the above sulfuric acid solution of 1-decanol was added under room temperature for about 15 minutes meanwhile a hard-to-stir material separates out. The reaction mixture was stirred under room temperature for another 26 hours then it was filtered, washed with methyl t-butyl ether and dried under vacuum at a temperature between 50-60° C. In this manner 3.96 g of product was obtained which was identified as polymorph Form I by analytical tests.

Yield: 80.9%.

Melting point: 181-182° C. (determined by DSC).

### EXAMPLE 15

### Preparation of polymorph Form I of (S)-(+)-methyl-α-(2-chlorophenyl)-6,7-dyhidro-thieno[3,2-c]pyridine-5(4H)-acetate hydrogen sulfate of formula I

Applying the procedure described in Example 14 the title product was obtained with the difference, that instead of the amounts of Example 14, 4 ml (3.3 g) of 1-decanol, 0.72 ml (96 wt %) of sulfuric acid, 4.0 g of (S)-(+)-clopidogrel base and 40.0 ml (29.6 g) of methyl t-butyl ether were used and the reaction mixture was stirred for another 24 hours. In this manner 3.27 g of product was obtained which was identified as polymorph Form I by analytical tests.

Yield: 62.6%.

Melting point: 182-183° C. (determined by DSC).

1. Process for the preparation of the pharmaceutically applicable polymorph Form I of (S)-(+)-methyl-α-(2-chlorophenyl)-6,7-dyhidro-thieno[3,2-4-pyridine-5(4H)-acetate hydrogen sulfate of formula I 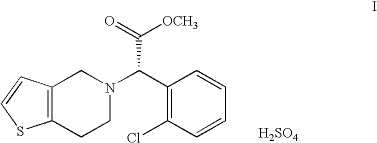
by reacting (S)-(+)-methyl-α-(2-chlorophenyl)-6,7-dyhidro-thieno[3,2-c]pyridine-5(4H)-acetate and sulfuric acid in the presence of solvents, the process comprising the steps of:
dissolving (S)-(+)-methyl-α-(2-chlorophenyl)-6,7-dyhidro-thieno[3,2-c]pyridine-5(4H)-acetate in an ether;
mixing this solution with a solution of a C₆-C₁₁ alcohol and sulfuric acid; and
recovering the so obtained compound of formula I from the mother liquor.

2. The process according to claim 1, wherein one or more straight or branched chain aliphatic ether of general formula Cₙ—O—Cₘ is used wherein n and m may be different or identical and n is 1-4 and m is 2-4.

3. The process according to claim 2, wherein methyl-t-butyl ether is used.

4. The process according to any one of claims 1-3, wherein one or more linear or branched, aliphatic or cyclic, primary, secondary or tertiary C₆-C₁₁ alcohol is used.

5. The process according to claim 4, wherein 1-decanol is used.

6. The process according to claim 1 wherein the (S)-(+)-methyl-α-(2-chlorophenyl)-6,7-dihydrothieno[3,2-c]pyridine-5(4H)-acetate is dissolved in 0.7-7.4fold amount of ether.

7. The process according to claim 6, wherein the (S)-(±)-methyl-α-(2-chlorophenyl)-6,7-dihydrothieno[3,2-c]pyridine-5(4H)-acetate is dissolved in 7.4fold amount of ether.

8. The process according to any one of claims 1-7, wherein the alcohol is used in a 0.8-4.1fold amount based on the weight of the (S)-(+)-methyl-α-(2-chlorophenyl)-6,7-dihydrothieno[3,2-c]pyridine-5(4H)-acetate.

9. The process according to claim 8, wherein the alcohol is used in an 1.66fold amount based on the weight of (S)-(+)-methyl-α-(2-chlorophenyl)-6,7-dihydrothieno[3,2-c]pyridine-5(4H)-acetate.

10. The process according toy claim 1 wherein the ether is used in a 0.17-8.96fold amount based on the amount of the alcohol.

11. The process according to claim 10, wherein the ether is used in a 4.45fold amount based on the amount of the alcohol.

12. The process according to claim 1 wherein 0.9-1.25 mol equivalents sulfuric acid is used per one mol of (S)-(+)-methyl-α-(2-chlorophenyl)-6,7-dihydrothieno[3,2*c]pyridine-5(4H)-acetate.

13. The process according to claim 12, wherein 1 mol equivalent of sulfuric acid is used per one mol of (S)-(+)-methyl-α-(2-chlorophenyl)-6,7-dihydrothieno[3,2-c]pyridine5(4H)-acetate.

14. The process according to claim 1 wherein the sulfuric acid is employed in a concentration of 90-100 wt/%.

15. The process according to claim 14, wherein the sulfuric acid is employed in a concentration of 96 wt/%.

16. The process according to claim 1 wherein the crystallization is carried out for 24-48 hours.

17. The process according to claim 1 wherein the reaction is carried out at room temperature.
